SEQUENCE LISTING (39 sequences)

SEQ ID NO: 1 - protein, 495 aa, Arabidopsis thaliana
MVSETTKSSPLHFVLFPFMAQGHMIPMVDIARLLAQRGVIITIVTTPHNAARFKNVLNRAIESGLPINLVQVKFPYLEAGLQEGQENIDSLDTMERMIPFFKAVNFLEEPVQKLIEEMNPRPSCLISDFCLPYTSKIAKKFNIPKILFHGMGCFCLLCMHVLRKNREILDNLKSDKELFTVPDFPDRVEFTRTQVPVETYVPAGDWKDIFDGMVEANETSYGVIVNSFQELEPAYAKDYKEVRSGKAWTIGPVSLCNKVGADKAERGNKSDIDQDECLKWLDSKKHGSVLYVCLGSICNLPLSQLKELGLGLEESQRPFIWVIRGWEKYKELVEWFSESGFEDRIQDRGLLIKGWSPQMLILSHPSVGGFLTHCGWNSTLEGITAGLPLLTWPLFADQFCNEKLVVEVLKAGVRSGVEQPMKWGEEEKIGVLVDKEGVKKAVEELMGESDDAKERRRRAKELGDSAHKAVEEGGSSHSNISFLLQDIMELAEPNN

SEQ ID NO: 2 - protein, 495 aa, Vigna angularis
MVLQTNNVPHFVLFPMMAQGHIIPMMDIARILAQRGVIITVFTTPKNASRFNSVISRAVSSGLKIRLVQLNFPSKEAGLREGCENLDMVSSNDMSKIFQVIHMPQKPAEEFFQTLTPKPSCIISDFCIAWTLQLAEKYHIPRVSFHGFSCFCLHCRYVIHTSDFCRSITSESKYFTIPGIPDKIQVTKEQLPGSLATDLDDFKDQVRDAEKKSYGVIVNTFWRVGEGICEGFSRRLKNNKAWFIGPVSLCNKDGLDKAQRGKQASINENHCLKWLDVQQAKSVVYVCFGSICNLIPSQLVELALALEDTKRPFVWVIREGSQLQELEKWFSEEGFEERTKGRGLIIGGWAPQVMILSHPSIGGFLTHCGWNSTLEGICAGVPLVTWPLFGDQFLNEKPVSDVLRIGVSVGAEVPLKWGEEEKRGVMVKKDDIKRAICMVMDDEEGKERRERVCKLSEMGKRAVEEGGSSHLDVTLLIQDIMQQTNTKEDESLSDV

SEQ ID NO: 3 - protein, 476 aa, Nicotiana tabacum
MGQLHFFFFPVMAHGHMIPTLDMAKLFASRGVKATIITTPLNEFVFSKAIQRNKHLGIEIEIRLIKFPAVENGLPEECERLDQIPSDEKLPNFFKAVAMMQEPLEQLIEECRPDCLISDMFLPWTTDTAAKFNIPRIVFHGTSFFALCVENSVRLNKPFKNVSSDSETFVVPDLPHEIKLTRTQVSPFERSGEETAMTRMIKTVRESDSKSYGVVFNSFYELETDYVEHYTKVLGRRAWAIGPLSMCNRDIEDKAERGKKSSIDKHECLKWLDSKKPSSVVYVCFGSVANFTASQLHELAMGIEASGQEFIWVVRTELDNEDWLPEGFEERTKEKGLIIRGWAPQVLILDHESVGAFVTHCGWNSTLEGVSGGVPMVTWPVFAEQFFNEKLVTEVLKTGAGVGSIQWKRSASEGVKREAIAKAIKRVMVSEEADGFRNRAKAYKEMARKAIEEGGSSYTGLTTLLEDISTYSSTGH

SEQ ID NO: 4 - protein, 476 aa, Nicotiana tabacum
MGQLHIFFFPVMAHGHMIPTLDMAKLFASRGVKATIITTPLNEFVFSKAIQRNKHLGIEIEIRLIKFPAVENGLPEECERLDQIPSDEKLPNFFKAVAMMQEPLEQLIEECRPDCLISDMFLPWTTDTAAKFNIPRIVFHGTSFFALCVENSVRLNKPFKNVSSDSETFVVPDLPHEIKLTRTQVSPFERSGEETAMTRMIKTVRESDSKSYGVVFNSFYELETDYVEHYTKVLGRRAWAIGPLSMCNRDIEDKAERGKKSSIDKHECLKWLDSKKPSSVVYICFGSVANFTASQLHELAMGVEASGQEFIWVVRTELDNEDWLPEGFEERTKEKGLIIRGWAPQVLILDHESVGAFVTHCGWNSTLEGVSGGVPMVTWPVFAEQFFNEKLVTEVLKTGAGVGSIQWKRSASEGVKREAIAKAIKRVMVSEEADGFRNRAKAYKEMARKAIEEGGSSYTGLTTLLEDISTYSSTGH

SEQ ID NO: 5 - protein, 497 aa, Oryza sativa
MSSAGHAVDQQRKSTTMKAHFVLVPMMAQGHMIPMTGMARLLAEHGAQVSFVTTPVNAARMAGFVTAVEAAGLAVQLVKLPFPATEFGLPDGCENLDMIQSRDLSRNFMEACGALREPLTARLRQLCPPPSCIISDMVQWWTGEIARELGIPRLTFDGFCTFASLARYIIFRDKLLDNVADEEIVTFSGFPMLLELPKARCPGSLCVPGMEQIRDKMYEEELQSDGNVMNSFQELETLYIESFEQITGKKVWTIGPMCLCDRDSNMMAARGNKASVDEAKCLQWLDSKKPGSVIFVSFGSLASTAPQQLVELGLGLEASKEPFIWVIKAGNKFPEVEEWLADGFEERVKDRGMIIRGWAPQVMILWHQAIGGFMTHCGWNSTIEGICAGVPMITWPHFAEQFLNEKFVVNLLKIGLEIGVKGVAQWGSEHKEVRVTRNAVETAVSTLMNDGEAAQEMRMRAKDLGVKARRALEEGGSSYDNISLLIQEMGNKQNASG

SEQ ID NO: 6 - protein, 466 aa, Lycopersicon esculentum
HFFFFPDDAQGHMIPTLDMANVVACRGVKATIITTPLNESVFSKAIERNKHLGIEIDIRLLKFPAKENDLPEDCERLDLVPSDDKLPNFLKAAAMMKDEFEELIGECRPDCLVSDMFLPWTTDSAAKFSIPRIVFHGTSYFALCVGDTIRRNKPFKNVSSDTETFVVPDLPHEIRLTRTQLSPFEQSDEETGMAPMIKAVRESDAKSYGVIFNSFYELESDYVEHYTKVVGRKNWAIGPLSLCNRDIEDKAERGRKSSIDEHACLKWLDSKKSSSIVYVCFGSTADFTTAQMQELAMGLEASGQDFIWVIRTGNEDWLPEGFEERTKEKGLIIRGWAPQSVILDHEAIGAFVTHCGWNSTLEGISAGVPMVTWPVFAEQFFNEKLVTEVMRSGAGVGSKQWKRTASEGVKREAIAKAIKRVMASEETEGFRSRAKEYKEMAREAIEEGGSSYNGWATLIQDITSYR

SEQ ID NO: 7 - protein, 489 aa, Dorothenathus bellidiformis
MGTHSTAPDLHVVFFPFLAHGHMIPSLDIAKLFAARGVKTTIITTPLNASMFTKAIEKTRKNTETQMEIEVFSFPSEEAGLPLGCENLEQAMAIGANNEFFNAANLLKEQLENFLVKTRPNCLVADMFFTWAADSTAKFNIPTLVFHGFSFFAQCAKEVMWRYKPYKAVSSDTEVFSLPFLPHEVKMTRLQVPESMRKGEETHFTKRTERIRELERKSYGVIVNSFYELEPDYADFLRKELGRRAWHIGPVSLCNRSIEDKAQRGRQTSIDEDECLKWLNSKKPDSVIYICFGSTGHLIAPQLHEIATALEASGQDFIWAVRGDHGQGNSEEWLPPGYEHRLQGKGLIIRGWAPQVLILEHEATGGFLTHCGWNSALEGISAGVPMVTWPTFAEQFHNEQLLTQILKVGVAVGSKKWTLKPSIEDVIKAEDIEKAVREVMVGEEGEERRRRAKKLKEMAWRAIEEGGSSYSDLSALIEELKGYHTSEKE

SEQ ID NO: 8 - DNA, 30 bp, Artificial
  Description of Artificial SequenceDNA
actaagcttggaatcatggtttccgaaaca

SEQ ID NO: 9 - DNA, 25 bp, Artificial
  Description of Artificial SequenceDNA
aagcggccgcatactcaattattgg

SEQ ID NO: 10 - DNA, 31 bp, Artificial
  Description of Artificial SequenceDNA
ctaagcttggaatcatggcatcggaatttcg

SEQ ID NO: 11 - DNA, 30 bp, Artificial
  Description of Artificial SequenceDNA
tagcggccgcattcatttcttgggttgttc

SEQ ID NO: 12 - DNA, 32 bp, Artificial
  Description of Artificial SequenceDNA
ctaagcttggaatcatggctttcgagaagacc

SEQ ID NO: 13 - DNA, 30 bp, Artificial
  Description of Artificial SequenceDNA
tagcggccgcattcaactcttggattctac

SEQ ID NO: 14 - DNA, 32 bp, Artificial
  Description of Artificial SequenceDNA
ctaagcttggaatcatggctacggaaaaaacc

SEQ ID NO: 15 - DNA, 29 bp, Artificial
  Description of Artificial SequenceDNA
tagcggccgcattcattcttgaattgtgc

SEQ ID NO: 16 - DNA, 31 bp, Artificial
  Description of Artificial SequenceDNA
ctaagcttggaatcatggcttccgaaaaatc

SEQ ID NO: 17 - DNA, 29 bp, Artificial
  Description of Artificial SequenceDNA
tagcggccgcattcagttcttggatttca

SEQ ID NO: 18 - DNA, 31 bp, Artificial
  Description of Artificial SequenceDNA
ctaagcttggaacatgtgttctcatgatcct

SEQ ID NO: 19 - DNA, 30 bp, Artificial
  Description of Artificial SequenceDNA
tagcggccgcattcaattattggactgtgc

SEQ ID NO: 20 - DNA, 59 bp, Artificial
  Description of Artificial SequenceDNA
ggatgcccgaacaaaagttaatttcagaagaggacttatcaaagcttgaggcctcgcga

SEQ ID NO: 21 - DNA, 26 bp, Artificial
  Description of Artificial SequenceDNA
tacaagcgaaatcgccaagaagttca

SEQ ID NO: 22 - DNA, 26 bp, Artificial
  Description of Artificial SequenceDNA
cttcttggcgatttcgcttgtataag

SEQ ID NO: 23 - DNA, 30 bp, Artificial; identical to SEQ ID NO: 8
  Description of Artificial SequenceDNA

SEQ ID NO: 24 - DNA, 25 bp, Artificial
  Description of Artificial SequenceDNA
tcttgtgaattcaactctatcagga

SEQ ID NO: 25 - DNA, 27 bp, Artificial
  Description of Artificial SequenceDNA
tacaagcaaaatcgccaagaagttcaa

SEQ ID NO: 26 - DNA, 25 bp, Artificial
  Description of Artificial SequenceDNA
acttcttggcgattttgcttgtatt

SEQ ID NO: 27 - DNA, 31 bp, Artificial
  Description of Artificial SequenceDNA
taagcttggaatcatgtgttctcatgatcct

SEQ ID NO: 28 - DNA, 26 bp, Artificial
  Description of Artificial SequenceDNA
tcacccgggaaacagtaatcatgtcc

SEQ ID NO: 29 - DNA, 22 bp, Artificial
  Description of Artificial SequenceDNA
cgaggcagatcgtcagtcagtc

SEQ ID NO: 30 - DNA, 19 bp, Artificial
  Description of Artificial SequenceDNA
atccggggttgaacagcct

SEQ ID NO: 31 - DNA, 19 bp, Artificial
  Description of Artificial SequenceDNA
tcaattattgggttctgcc

SEQ ID NO: 32 - DNA, 19 bp, Artificial
  Description of Artificial SequenceDNA
ggagaaaataggagtgtta

SEQ ID NO: 33 - DNA, 19 bp, Artificial
  Description of Artificial SequenceDNA
tcagttcttggatttcact

SEQ ID NO: 34 - DNA, 18 bp, Artificial
  Description of Artificial SequenceDNA
gagaaactggtcgtacaa

SEQ ID NO: 35 - DNA, 19 bp, Artificial
  Description of Artificial SequenceDNA
tcaattattggactgtgct

SEQ ID NO: 36 - DNA, 21 bp, Artificial
  Description of Artificial SequenceDNA
gtccttctttctggtaaacgt

SEQ ID NO: 37 - DNA, 21 bp, Artificial
  Description of Artificial SequenceDNA
aacccttgaggttgaatcatc

SEQ ID NO: 38 - DNA, 38 bp, Artificial
  Description of Artificial SequenceDNA
gttaaaagcttacatgtgcattacggtctgtgtgaata

SEQ ID NO: 39 - DNA, 38 bp, Artificial
  Description of Artificial SequenceDNA
tttcggatcccatgattcaaccttagtaagaaactctc

(19) United States
(12) Patent Application Publication
(10) Pub. No.: US 2006/0183202 A1
(43) Pub. Date: Aug. 17, 2006

(54) Method for detoxification of mycotoxins

(75) Inventors: Brigitte Poppenberger, York (GB); Gerhard Adam, Vienna (AT); Franz Berthiller, Vienna (AT); Rudolf Krska, Tulln (AT); Karl Kuchler, Vienna (AT); Christian Luschnig, Vienna (AT); Josef Glossl, Vienna (AT); Doris Lucyshyn, Vienna (AT); Rainer Schuhmacher, Tulln (AT); Tobias Sieberer, York (GB)

Correspondence Address: FULBRIGHT & JAWORSKI L.L.P., 600 CONGRESS AVE., SUITE 2400, AUSTIN, TX, 78701, US

(73) Assignee: Universtat fur Bodenkultur Wien, Vienna (AT)

(21) Appl. No.: 10/570,032

(22) PCT Filed: Sep. 1, 2004
(86) PCT No.: PCT/EP04/09728

§ 371 Date: Feb. 28, 2006

(30) Foreign Application Priority Data
Sep. 1, 2003 (EP) 03450194.0

(51) Int. Cl. A01H 1/00; C12P 19/62; C12N 5/04

(52) U.S. Cl. 435/76; 435/419; 800/279

(57) ABSTRACT

Disclosed is a method for detoxification of mycotoxins wherein mycotoxin is contacted with a glucosyltransferase in the presence of an activated glucose.

The present invention relates to a method for detoxification of mycotoxins as well as a method for producing derivatives of mycotoxins.

A complex of closely related species of the genus Fusarium is responsible for destructive and economically very important diseases of cereal crops (Fusarium head blight, FHB, of wheat and barley) and maize (Fusarium ear rot). In years with climatic conditions that favor the development of the fungi, Fusarium infections can reach epidemic proportions (1). Diseases caused by the plant pathogens do not only severely reduce yield, but also result in contamination of grain with unacceptable high amounts of mycotoxins, a problem of world-wide significance. A toxin class of particular concern to human and animal health are trichothecenes, sesquiterpenoid epoxides which are potent inhibitors of eukaryotic protein synthesis. More than 180 compounds of this class have been isolated from natural sources, predominantly from Fusarium species (2). Depending on the concentration and the substitution pattern of the trichothecene, either translational initiation, elongation or termination are preferentially inhibited (3).

F. graminearum and F. culmorum are the two most relevant causative agents of FHB of cereals. Whereas F. graminearum lineages present in Europe and North America produce predominantly deoxynivalenol (DON), and the acetylated derivatives 3-acetyl-deoxynivalenol (3-ADON) and 15-acetyl-deoxynivalenol (15-ADON), producers of nivalenol (NIV), which contains one additional hydroxyl group (FIG. 1A), dominate in Asia (4). While the toxicity of these trichothecenes is well studied in animal systems (5), little is known about differences in phytotoxicity. Animal exposure to DON (which is also known as vomitoxin) has been shown to cause adverse health effects, the neural and immune system being the most sensitive targets (6). In contrast to high doses of DON, which inhibit antibody production, low doses of DON act synergistically with bacterial lipopolysaccharide stimulating proinflamatory processes (6). To protect consumers, advisory levels for food have been established by the US Food and Drug Administration, and recently action levels for DON have been recommended by the European Community (7).

The role of DON in plant disease is still matter of discussion (8), but most of the evidence is in favor of a function as virulence factor. Fusarium mutants with a disrupted gene involved in trichothecene biosynthesis (trichodiene synthase, Tri5) are still pathogenic, however they exhibit reduced virulence on wheat (9) due to their inability to spread from the infection site (10). DON can move ahead of the fungus in infested plants, suggesting a role in conditioning host tissue for colonization (11). Results of several studies indicate that the in vitro resistance of wheat cultivars towards DON correlates with FHB resistance in the field (12), which is also the case for segregating experimental populations.

Increased resistance against toxic substances can be caused by several mechanisms, ranging from reduced uptake to bypass, over-expression or mutation of the toxin target. Yet, a very prominent process seems to be metabolic transformation often followed by compartmentation (13). An observed decline in the concentration of DON, which occurred in Fusarium infected wheat in the field (14) suggested that the toxin may be metabolized.

Acetylation of trichothecenes (e.g. DON) was suggested as a potential resistance strategy (U.S. Pat. No. 6,346,655 B1), however, the resulting acetylated trichothecenes (e.g. Acetyl-DON=ADON) is equivalent in its overall toxicity to the non-acetylated form (e.g. DON), as proven by Eudes et al. in a coleoptile elongation test (53). Therefore, acetylation does not result in a reduction of trichothecene toxicity.

Two wheat cultivars differing in Fusarium resistance showed differences in their ability to form a DON metabolite, which was suspected to be a glucoside (15). Sewald et al. (16) incubated maize suspension culture with radiolabeled DON and proved that it was primarily conjugated to 3-β-D-glucopyranosyl-4-deoxynivalenol. No plant enzymes capable of modifying the trichothecenes or other mycotoxins by transfer of a sugar moiety have been described so far.

Genome sequencing projects revealed the existence of a vast number of genes in plants that code for putative UDP-glycosyltransferases (UGT), predicted to conjugate small molecules. For instance, Arabidopsis thaliana has been shown to harbor more than 100 members of this multigene family (17) whose functions are largely unknown.

It is therefore an object of the present invention to provide a method for detoxification of mycotoxins.

Suitable preparations of mycotoxins or derivatives thereof, especially sugar-conjugate derivatives of mycotoxins are often difficult to provide. Since stereoselectivity is crucial to this kind of products, purely chemical syntheses based on common bulk mycotoxins are often diffcult, if not impossible, at least on an industrial scale. Another object of the present invention is therefore to provide methods for synthesising sugar-conjugate derivatives of mycotoxins.

Therefore the present invention provides a method for detoxification of mycotoxins wherein a mycotoxin is contacted with a glucosyltransferase in the presence of an activated glucose.

As mentioned above, there has not been any report being published in the prior art with respect to (plant) enzymes being capable of modifying mycotoxins by transfer of a sugar molecule, and of course not with respect to such enzymes being able to detoxify such mycotoxins, i.e. reducing the toxicity of mycotoxins to a considerable extent, e.g. by more than 80%, preferably more than 90%, especially more than 95%, as determined by specific mycotoxin assays, such as inhibition of in vitro protein synthesis.

According to the present invention it has been found that glucosyltransferases are very specific tools for detoxification of mycotoxins. Specificity is on the one hand given by a certain specificity of given glucosyltransferases to only a limited number of mycotoxins and on the other hand also specificity with respect to the mode of detoxification, namely by site specific glucosylation of the mycotoxins. While this specificity limits the scope of application of a given single glucosyltransferase, the present invention provides tools for easily determining the specificities of any glucosyltransferase with respect to any mycotoxin, especially trichothecene mycotoxins. Therefore, if a given enzyme is specific for a given set of mycotoxins, the present invention allows straightforward identification of specificities for other glucosyltransferases and mycotoxins.

Since uridine diphosphate glucose (UDP-glucose, e.g. glucose which is produced by enzymes, such as pyrophosphorylases) is a preferred activated biological form of glucose, a preferred embodiment of the present method uses UDP-glucosyltransferase as glucosyltransferase and an UDP-glucose as activated glucose (=cosubstrate), however, any form glucose, which can be utilised by a glucosyltransferase (e.g. ADP-glucose, CDP-glucose, or any other form of biologically and synthetically activated glucose being suitable substrates for transfer to mycotoxins by glucosyltransferases) can be used according to the present invention.

A glucosyltranferase according to the present invention is defined as a (naturally) occuring or synthetically (recombinantly) designed or produced enzyme which is capable (as its main or as a side activity) of transferring a glucose moiety to a substrate molecule using an activated glucose as co-substrate thereby producing a glucosylated substrate molecule.

Preferred mycotoxins to be detoxified by the method according to the present invention are—due to their economic importance—trichothecenes, especially trichothecenes having a free hydroxy-group at position C₃ and two hydrogen groups at C₄. Especially the latter trichothecenes are specifically glucosylated either at their position 3 or—if present—at a free hydroxy group at position 7 or at both positions. Therefore, preferred mycotoxins are deoxynivalenol, 15-acetyl-deoxynivalenol, 15-acetoxyscirpendiol or mixtures thereof.

A preferred way of performing the method according to the present invention is the in vivo mode. This can be done using recombinant DNA technology and/or glucosyltransferase expression enhancing compounds. Preferred examples comprise expression of a glucosyltransferase in a transgenic plant cell (or plant tissue or whole plant) containing a recombinant glucosyltransferase and/or a recombinant regulating region for a glucosyltransferase, especially a mycotoxin-inducible promoter, especially of a glucosyltransferase. Preferred examples for glucosyltransferase expression enhancing compounds are substances which up-regulate glucosyltransferases, e.g. toxins (if expression of the transferase is controlled by toxin-inducible promoters). In principle, any cell which can be genetically manipulated so that it expresses a recombinant (i.e. a gene which has been artificially introduced into this cell or its precursor and which naturally is not present at this site of the genome of the cell) plant glucosyltransferase gene can be used for this purpose. Preferred embodiments are transgenic bacteria, yeasts, baculovirus infected insect cells, etc. expressing a plant glucosyltransferase gene.

In an industrial process for detoxification of mycotoxins, the glucosyltransferase can advantageously be immobilised (according to standard immobilisation techniques) to a solid surface and a mycotoxin containing solution or suspension can then be contacted with this immobilised glucosyltransferase.

Preferred examples of glucosyltransferases to be used according to the present invention are UDP-glucosyltransferases corresponding to subfamily 73C of Arabidopsis thaliana, especially UDP-glucosyltransferases 73C4 and 73C5.

According to another aspect, the present invention enables a method for producing glucosylated mycotoxins wherein a mycotoxin is contacted with a glucosyltransferase in the presence of an activated glucose. Therewith, the above mentioned enzyme specificities can be properly used for producing specifically glucosylated mycotoxin derivatives. If a mycotoxin is glucosylated in a non-enzymatic (“chemical”) way, a crude mixture of glucosylation products is obtained (each free hydroxy group is in principle an acceptor site for glucosyl residues). Acetylation as protecting means for such groups has also its drawbacks with respect to specificity (also all hydroxy groups are acetylated). In using substrate and site specificity, the present invention provides a method for producing well defined glucosylated derivatives. If more than one hydroxy group is glucosylated or if more than one position is capable of being glucosylated by a given glucosyltransferase, such a limited number of products (e.g. 2 to 5) may easily be separated by suitable separation means such as HPLC (for example, DON, being glucosylated at position number 3, is easily separatable from DON, being glucosylated at position 7).

The preferred method steps described above are also applicable for this aspect of the invention.

According to another aspect, the present invention relates to transgenic plants or a transgenic (plant) cells containing a recombinant glucosyltransferase and/or a recombinant expression regulating element, especially a promoter region for a glucosyltransferase. These plants or (plant) cells may be used in a method according to the present invention.

The present invention also relates to a genetically modified cell or organism, especially a plant or plant cell, comprising a non-naturally occurring (transgenic) glucosyltransferase in its genome and being mycotoxin resistant, especially trichothecene resistant, due to the presence of such a glucosyltransferase as transgene. Alternatively, an endogeneous glucosyltransferase in a cell may be subjected to a different, transgenic expression regulating region or element, such as a promoter, leading to an enhanced expression of the endogeneous glucosyltransferase (i.e. an enhanced expression activity of a homologous glucosyltransferase due to transgenic promoters). Also such cells show enhanced resistance against mycotoxins as described herein. Such cells and plants can be produced by applying standard methods to the teachings of the present invention (see e.g. FIG. 6B and the examples as well as e.g. (54)).

Preferred cells according to the present invention are also yeast cells. Since yeast cells may have a limited uptake capability, preferred yeast cells according to the present invention are designed or selected for an increased mycotoxin-uptake capability compared to the wild type or starting strain. Specifically preferred according to the present invention are yeasts in which one or more, preferably three or more, ABC transporters are reduced or inactivated in their activity, especially pdr5. Such ABC transporter mutants are characterized by an enhanced uptake of mycotoxins. Preferably, the cells according to the present invention have a deletion in pdr5, pdr10, pdr15, snq2, yor1, ayt1 or combinations of these deletions.

These cells may preferably be used for detoxification of mycotoxin containing solutions or suspensions, especially for detoxification of trichothecenes in agriculture and beer production; and in the production of glucosylated mycotoxins, especially glucosylated DON or 15-ADON.

The present invention is further illustrated by the following examples and the figures, yet without being restricted thereto.

### FIGURES

FIG. 1. Spectrum of trichothecene resistance of yeast expressing Arabidopsis thaliana UDP-glucosyltransferases of subfamily UGT73C. A, Structure and numbering of the ring system of class B trichothecenes used for resistance testing. The different residues at variable positions R1-R4 are indicated in the table (OAc: —OCOCH₃, OBe: —OCOCH═CHCH₃ (Z)). The relevant R1 is underlined (OH in DON, already blocked by acetylation in 3-ADON). Increased resistance conferred by DOGT1 expression is indicated by plus, lack of protection by minus. B, Genomic organisation of the UGT gene cluster on chromosome II (subfamily 73C) containing DOGT1 (UGT73C5, locus At2g36800). The DON detoxifying DOGT1 and 73C4 are shaded in gray. C, Yeast strains were spotted on YPD plates containing the indicated amount of toxin (ppm: mg/l). The strains are wild-type YZGA515 without plasmid (wt), this strain transformed with empty vector (c), and the transformants expressing the myc-tagged 73C UGTs (C5=DOGT1, underlined, spotted in duplicate). Resistance is conferred by plasmids leading to overexpression of C5 and C4. D, Western blot analysis of the yeast strains used for resistance testing. The N-terminal c-Myc epitope-tag introduced into the respective 73C UGT family members is detected (C5=DOGT1, underlined). A transformant containing the empty expression vector was used as control.

FIG. 2. Amino acid alignment of plant UDP-glucosyltransferases with high amino acid similarity to DOGT1. The regions implicated (Ref. 38) in acceptor substrate binding (dottet) and the UGT consensus sequence motif (dashed) are indicated by boxes below the sequences. The triangle above the sequence in the hypothetical acceptor binding region marks the lysine 136 in DOGT1 which has been altered by in vitro mutagenesis. The genbank accession numbers of the predicted proteins are: ADGT-9, glucosyltransferase-9 of Vigna angularis (AB070752); TOGT1, Phenylpropanoid:glucosyltransferase 1 of Nicotiana tabacum (AF346431); IS5a of Nicotiana tabacum (U32644); putative glucosyltransferase of Oryza sativa (AP002523); Twil of Lycopersicon esculentum (X85138); Betanidin-5-O-glucosyltransferase of Dorothenathus bellidiformis (Y18871).

FIG. 3. DOGT1 expression is developmentally regulated and induced by DON and stress response related compounds. A, GUS-staining of seedlings homozygous for a transcriptional DOGT1 promoter-GUS fusion. Upper row: three days after germination (3 DAG) the expression of the fusion protein is restricted to the vasculature of root and hypocotyl and the meristematic region of the root tip. Middle: Later in development (10 DAG) staining in the root vasculature diminishes except for regions where lateral roots are formed. Lower row: In aerial parts of adult plants DOGT1 expression is restricted to petals of flowers and abscission zones. B, DON treatment (5 ppm for 4 h) of seedlings (14 DAG) expressing the translational GUS-fusion induces expression of the GUS reporter. Both samples were stained for 2 hours. C, Semiquantitative reverse transcriptase PCR analysis of induction of expression of DOGT1, UGT73C4 and UGT73C6 following treatment with DON (5 ppm), SA (100 μM), JA (50 μM) and ACC (2 μM). UBQ5 was used as an internal control.

FIG. 4. The N-terminal part of DOGT1 is essential for its ability to detoxify DON. A, Amino acid alignment of DOGT1 and its closest homologue UGT73C6, which is not protecting against DON (black: amino acids identity). The conserved EcoRI restriction site was used to generate chimaeric UGTs consisting of the N-terminus of one and the C-terminus of the respective other gene. B, Yeast transformants were spotted in duplicate on YPD medium containing the indicated concentrations of DON. Upper left: transformants expressing the chimaeric enzyme consisting of the UGT73C6 N-terminus and DOGT1 C-terminus (short: C6-DOG). Upper right: Strains expressing the chimaeric protein consisting of DOGT1 N-terminus and UGT73C6 C-terminus (DOG-C6). Lower row: transformants expressing resistance conferring DOGT1 (left), and inactive 73C6 (right). The hybrid containing the N-terminal part of DOGT1 is resistant to higher DON concentrations than the strain expressing the other hybrid (C6-DOG) or UGT73C6.

FIG. 5. Fragmentation pattern of synthesized DON-glucosides and a DON-metabolite formed in yeast cells expressing DOGT1 in mass spectrometry. A, The synthesized DON-3-O-glucoside yields a fragment of 427.2 m/z in the linear ion trap (MS/MS/MS). B, this fragment was not produced by the synthesized DON-15-O-glucoside sample as a separation of the —CH₂OH group at C₆ is prevented by the glucose moiety present at the hydroxyl group. C, The DON-metabolite formed in yeast expressing DOGT1 eluted in the HPLC with the same retention time as the DON-3-O-glucoside and showed the fragmentation pattern corresponding to the DON-3-O-glucosid reference substance.

FIG. 6. glucosylation of DON reduces toxicity in vitro and in vivo. A, Structure of the proposed reaction product: DOGT1 catalyses the transfer of glucose from UDP-glucose to the 3-OH position of DON. B, Comparison of the inhibition of in vitro protein synthesis of wheat ribosomes by DON and DON-3-O-glucoside, determined using a wheat germ extract based coupled transcription/translation system. Luminescence was measured and expressed as % luciferase activity of control samples without toxin. C, Western blot analysis of A. thaliana lines homozygous for an overexpression construct encoding c-Myc-tagged DOGT1. A schematic drawing of the plant transformation vector is shown below (GENT: gentamycin resistance used as selectable marker; 2×35S duplicated promoter of cauliflower 33S transcript (see Experimental Procedures for details)). Col-0 was used as a negative control. 2 lines with high and 2 lines with low levels of c-Myc-DOG1 protein are shown. D, Seed germination on DON containing MS medium. Compared to wild-type (Col-0) transgenic Arabidopsis lines expressing high amounts of DOGT1 (e.g. line 1319/2) exhibit enhanced resistance.

### EXAMPLES

For illustrating the present invention, in the examples hereinafter the cloning of an Arabidopsis UGT by functional expression in yeast and its characterization is described. This enzyme is able to detoxify the Fusarium mycotoxin DON and its acetylated derivative 15-ADON. The methods used in these examples are also directly applicable to other glucosyltransferases and mycotoxins without undue experimental burden.

### EXPERIMENTAL PROCEDURES

Yeast strains. The yeast strains used in this work are derived from YPH499 (Mat a, ade2-101oc, his3-Δ200, leu2-Δ1, lys2-801a, trp1-Δ1, ura3-52) (18). The relevant genotype of YZGA452 is pdr5Δ::TRP1, pdr10Δ::hisG, snq2Δ::hisG, yor1Δ::hisG. YZGA515 (pdr5Δ::TRP1, pdr10Δ::hisG, pdr15Δ::loxP-KanMX-loxP, ayt1Δ::URA3) was constructed by disruption of the acetyltransferase AYT1 in strain YHW10515.

Plant material and growth conditions. A. thaliana experiments were conducted with the wild-type ecotype Columbia-0 (Col-0). For propagation seeds were sterilized, plated on standard MS growth medium (19) supplemented with 1.0 % sucrose and 1.0% phytagar (Life Technologies) and subjected to a 2 day dark treatment at 4° C. to synchronize germination. The seedlings were grown for 2 weeks in a controlled environment of 16 h/8 h light-dark cycle (140 μmol m⁻² sec⁻¹ white light) at 22° C. before they were transferred to soil and grown at 20° C. and 55% humidity under continuous white light.

Arabidopsis thaliana cDNA library screen in yeast. The ATP-binding cassette (ABC) transporter deficient Saccharomyces cerevisiae strain YZGA452, which is hypersensitive to DON, was transformed with an A. thaliana cDNA library constitutively expressed under the control of the phoshoglucerate kinase (PGK1) promoter (20). A total of 10⁷ transformants were selected on minimal medium lacking uracil and transferred to media containing 180 ppm DON, sufficient to completely inhibit growth of yeast transformed with the empty library plasmid. Colonies that showed resistance were isolated and from candidates that formed single colonies on toxin containing media, the plasmid dependency of the phenotype was tested by plasmid DNA preparation and retransformation of YZGA452. The NotI fragment containing the cDNA insert of the candidate (which was named DON glucosyltransferase 1, DOGT1) was subcloned into pBluescript SKII+ (Stratagene) and sequenced.

Constitutive expression and immunodetection of the DON-glucosyltransferase (DOGT1) and close homologues in yeast. The intronless open reading frames (ORFs) of DOGT1 (UGT73C5, locus At2g36800) and 5 of its closest homologues (UGT73C1, At2g36750; UGT73C2, At2g36760; UGT73C3, At2g36780; UGT73C4, At2g36770; UGT73C6, At2g36790) were PCR amplified (Triple Master PCR System, Eppendorf) from genomic DNA using gene specific primers containing flanking HindIII and NotI restriction sites at the 5′ and 3′ ends, respectively
(DOGT1, fw: 5′-ACTAAGCTTGGAATCATGGTTTCCGAAACA-3′, rv: 5′-AAGCGGCCGCATACTCAATTATTGG-3′; 73C1, fw: 5′-CTAAGCTTGGAATCATGGCATCGGAATTTCG-3′, rv: 5′-TAGCGGCCGCATTCATTTCTTGGGTTGTTC-3′;
73C2, fw: 5′-CTAAGCTTGGAATCATGGCTTTCGAGAAGACC-3′, rv: 5′-TAGCGGCCGCATTCAACTCTTGGATTCTAC-3′;
73C3, fw: 5′-CTAAGCTTGGAATCATGGCTACGGAAAAAACC-3′, rv: 5′-TAGCGGCCGCATTCATTCTTGAATTGTGC-3′;
73C4, fw: 5′-CTAAGCTTGGAATCATGGCTTCCGAAAAATC-3′, rv: 5′-TAGCGGCCGCATTCAGTTCTTGGATTTCA-3′;
73C6: 5′-CTAAGCTTGGAACATGTGTTCTCATGATCCT-3′, rv: 5′-TAGCGGCCGCATTCAATTATTGGACTGTGC-3′). The PCR products were cloned into the HindIII+NotI cloning sites of the yeast expression vector pYAK7 (PADH1-c-Myc-PDR5 LEU2 2μ), replacing the PDR5 gene. The vector pYAK7 was constructed by first inserting the double stranded linker 5′-GGATGCCCGAACAAAAGTTAATTTCAGAAGAGGACTTATCAAAGCTTGAGGCCTCGCGA into the SmaI site of vector pAD4Δ (21), thereby generating the N-terminal c-Myc epitope and a HindIII site, into which a genomic HindIII fragment containing the yeast PDR5 was inserted in frame.

The tagged UGT constructs were verified by sequencing and used to transform the yeast strain YZGA515. The empty vector (HindIII+NotI digested and religated pYAK7) was used as a control. Transformants were selected on minimal media lacking leucine. Exponentially growing cultures were diluted to OD600 0.05 and spotted on YPD media containing increasing concentrations of different trichothecenes. The toxins used were: DON, 3-ADON, 15-ADON, NIV, trichothecin (TTC), T-2 toxin, HT-2 toxin, diacetoxyscirpenol (DAS) and veruccarin A (VA). With the exception of DON, 3-ADON and NIV, which were obtained from Biopure Referenzsubstanzen GmbH (Tulln, Austria), mycotoxins were purchased from Sigma and stored at −20° C. dissolved in 70% ethanol.

For immunodetection, the extraction of proteins from yeast cells was performed as described by Egner et al. (22). Western blot analysis was conducted with a primary mouse anti c-Myc antibody (1:5000, clone 9E10, Invitrogen).

Domain shuffling. The ORFs including the N-terminal c-Myc tag of DOGT1 and its closest homologue UGT73C6 were isolated from the yeast expression vectors by SmaI+NotI digestion, cloned into vector pBluescript SKII+ (which was cut with XhoI and treated with Klenow enzyme, and subsequently digested with NotI). The resulting plasmids were digested with HindIII and a conserved EcoRI site present in both genes that cleaves DOGT1 at nucleotide position 565 (73C6 at 568). Hybrids were constructed by ligation of the N-terminal part of one gene to the C-terminal part of the other. The resulting genes were moved back into the yeast expression vector pYAK7 using the HindIII and NotI restriction sites. Yeast strain YZGA515 was used as a host to test the constructs for altered detoxification abilities.

Site directed mutagenesis. Mutations were constructed by overlap extension PCR (23) using overlapping mutant primers DOG-K136E-fw (5′-TACAAGCGAAATCGCCAAGAAGTTCA-3′) and DOG-K136E-rv (5′-CTTCTTGGCGATTTCGCTTGTATAAG-3′) and flanking primers DOGIpYAK7-fw-a (5′-ACTAAGCTTGGAATCATGGTTTCCGAAACA-3′) and DOG-EcoRI-rv (5′-TCTTGTGAATTCAACTCTATC AGGA-3′) for mutagenesis of DOGT1, and mutant primers 73C6-E137K-fw (5′-TACAAGCAAAATCGCC AAGAAGTTCAA-3′) and 73C6-E137K-rv (5′-ACTTCTTGGCGATTTTGCTTGTATT-3′) and flanking primers 73C6pYAK7-fw (5′-TAAGCTTGGAATCATGTGTTCTCATGATCCT-3′) and DOG-EcoRI-rv for 73C6 mutagenesis. The resulting PCR products were cloned as HindIII+EcoRI fragments into the corresponding genes present in vector pBluescript SKII+. After sequencing, the ORFs were moved as HindIII+NotI fragments back into the yeast expression vector pYAK7 (replacing the PDR5 gene) and the resulting plasmids were transformed into strain YZGA515 to analyze the detoxification properties of the engineered UGTs.

Synthesis of 3-β-D-Glucopyranosyl-4-deoxynivalenol and 15-β-D-Glucopyranosyl-4-deoxynivalenol. To obtain reference material for HPLC and TLC, DON-3-glucoside and DON-15-glucoside were synthesized in 2-step reactions. In the first step, 15-Acetyl-DON or 3-Acetyl-DON were modified with 1ββ-Bromo-1-deoxy-2,3,4,6-tetra-O-acetyl-α-D-glucopyranose (acetobromoglucose) in toluene with CdCO₃ as catalyst to yield the DON-glucoside-acetates (24). Gentle hydrolysis of the acetates to the glucosides was performed in the second step using a strong basic anion exchanger (DOWEX1×2-400, Aldrich, USA). After checking the progress of the reaction with TLC (mobile phase toluene/ethyl acetate 1:1, v/v), the DON-glucosides were cleaned up using flash chromatography over silica gel with 1-butanol/1-propanol/ethanol/water (2:3:3:1, v/v/v/v). Further purification of the substances was performed with HPLC, by means of a RP-18 Aquasil column (Keystone, Waltham, USA) using acetonitrile/water (10:90, v/v) at 22° C.

The synthesized DON-derivatives were characterized in the negative electrospray interface (ESI) mode. LC-MS/MS analysis was performed on a QTrap-LC-MS/MS system (Applied Biosystems, Foster City, USA) equipped with ESI and a 1100 Series HPLC system (Agilent, Waldbronn, Germany). Chromatographic separation was achieved on a 150 mm×4,6 mm i.d., 3 μm, Aquasil RP-18 column (Keystone, Waltham, USA) at 22° C. using methanol/water (28:72, v/v). The flow rate was set to 0.3 ml/min. The ESI interface was used in the negative ion mode at 400° C. with CUR 20 psi, GS1 30 psi, GS2 75 psi, IS −4200 V, DP −46 V, EP −9 V, CE −30 eV, CAD high, LIT fill time 50 ms, Q3 entry barrier 8 V.

Isolation and analysis of DON metabolites in vivo. To elucidate the chemical structure of DON metabolites that result from enzymatic transformation of the mycotoxin by DOGT1, a highly tolerant strain was constructed and the DON-metabolite was extracted from toxin treated cells for subsequent HPLC analysis.

The yeast strain YZGA515 was transformed with c-Myc-tagged DOGT1 under the control of the constitutive ADH1 promoter and in addition with plasmid pRM561. This plasmid contains a mutant version of the ribosomal protein L3 (RPL3) of Saccharomyces cerevisiae that substantially increases DON resistance when expressed in yeast. Transformants were selected on minimal media lacking leucine and adenine.

The obtained yeast strain was grown to an OD₆₀₀ 0.7 in selective medium (SC-LEU-ADE). Cells were transferred to YPD medium (10% glucose) supplied with additional adenine, grown for 2 h at 30° C., harvested by centrifugation and diluted to an OD₆₀₀ of 3.0 in YPD. DON was added to 5 ml of culture starting with 200 ppm DON. After 3 h the concentration was increased to 400 ppm, after 6 h to 600 ppm, and after 9 h to 1.000 ppm. The cells were incubated for further 15 h at 30° C., before they were harvested, washed three times with ice-cold water, extracted in 2.5 ml of methanol/water (4:1) and sonicated. After centrifugation the supernatant was filtered through a glass microfibre filter (Whatman 1822 025). 500 μl of the yeast extracts were concentrated to dryness under a constant stream of nitrogen and dissolved in 100 μl HPLC-grade water.

Heterologous expression of DOGT1 in Escherichia coli. The DOGT1 protein was expressed in Escherichia coli XL1-blue as a GST fusion. The DOGT1 gene was released from the yeast expression vector by HindIII digestion and Klenow fill in, followed by a NotI digest. The resulting fragment was cloned into the SmaI+NotI sites of the GST gene fusion vector pGEX-4T-3 (Amersham Pharmacia). Recombinant fusion protein was purified using glutathione-coupled Sepharose (Amersham Pharmacia) according to the manufacturer's instructions.

To test the effect of the N-terminal GST tag on activity, the gene encoding the fusion protein was PCR amplified using DNA polymerase with proof reading activity (Pfu polymerase, MBI) and the fusion protein specific primers GSTDOGpYAK7-fw (5′-TCACCCGGGAAACAGTAATCATGTCC-3′) and GSTDOGpYAK7-rv (5′-CGAGGCAGATCGTCAGTCAGTC-3′). The PCR product was cloned HindIII+NotI into the yeast expression vector pYAK7. DON-detoxification ability was tested by expression in YZGA515 and application to toxin containing media as described earlier.

Enzyme assays. The glucosyltransferase activity assay mix contained 1 μg of recombinant GST-fusion protein, 10 mM 2-mercaptoethanol, 50 mM Tris/HCl pH 7.0, 0.5 mM radioactive labeled UDP-[¹⁴C] glucose (4.4 *10³ cpm, NEN Life Science Products, USA), 0.01% BSA and 1 mM of acceptor substrate (dissolved in DMSO in 20 mM stock solutions). The reactions were carried out in volumes of 20 μl at 30° C. for 1 h, stopped by adding 2 μl trichloroacetic acid (240 mg/ml), frozen and stored at −20° C.

Analysis of reaction products was performed by TLC. An aliquot of each sample was spotted on a silica-gel plate (Kieselgel 60; Merck) and developed with a mixture of 1-butanol/1-propanol/ethanol/water (2:3:3:1, v/v/v/v). The intensity of each radioactive spot was determined using a phosphoimager (STORM 860 system, Molecular Dynamics). The plates were additionally stained with panisaldehyde (0.5% in methanol /H₂SO₄/acetic acid, 85:5:10, v/v/v).

Inhibition of wheat ribosomes in vitro. To analyze whether the 3-β-D-Glucopyranosyl-4-deoxynivalenol is less phytotoxic than its aglucon, a wheat germ extract coupled in vitro transcription/translation system (TNT Coupled Wheat Germ Extract, T3, Promega) was used. After performing transcription/translation reactions for 25 minutes according to the manufacturers instructions in the presence of either DON, purified DON glucoside (1 μM, 2.5 μM, 5 μM, 10 μM and 20 μM) or water as a control, the activity of the firefly luciferase reporter was determined (Luciferase Assay System, Promega) using a luminometer (Victor 2, Wallac).

Plant treatment with different stress response related compounds for expression analysis. For Reverse Transcription (RT)-PCR analysis of mRNA expression of DOGT1 following treatments with DON, salicylic acid (SA), jasmonic acid (JA) and 1-aminocyclopropyl-carbonic acid (ACC) seedlings were grown for 2 weeks on vertical MS plates (0.8% phytagar) before they were transferred to liquid MS media. The plants were incubated for 48 h on an orbital shaker (50 rpm) before adding 5 ppm DON, 200 μM SA, 2 μM ACC or 50 μM JA. The compounds were kept in stock solutions dissolved either in 70% ethanol or in DMSO. Treatments with ethanol and DMSO were performed as controls. Plants were harvested at different time points, ground in liquid nitrogen and stored at −70° C. until RNA extraction was performed.

Analysis of mRNA expression of DOGT1 and close homologous by Reverse Transcriptase-PCR. Total RNA was isolated from plant tissue ground in liquid nitrogen with Trizol Reagent as recommended by the manufacturers (Gibco BRL Life Technologies). RNA was quantified photometrically and visually on a denaturing RNA gel analyzing 5 μg of total RNA.

cDNA was synthesized from 1 μg of total RNA (digested with DNa-seI) with 500 ng of a 18-mer oligo(dT) and the reverse transcriptase SuperScript (Gibco BRL Life Technologies). PCR was performed with approximately 2 μl of the 1:20 diluted cDNA using primers that amplify 200 to 400 bp large fragments located in the C-terminal part of the genes to be analyzed (DOGRT-fw: 5′-ATCCGGGGTTGAACAGCCT-3′, DOGRT-rv: 5′-TCAATTATTGGGTTCTGCC-3′; 73C4RT-fw: 5′-GGAGAAAATAGGAGTGTTA-3′, 73C4RT-rv: 5′-TCAGTTCTTGGATTTCACT-3′; 73C6RT-fw: 5′-GAGAAACTGGTCGTACAA-3′, 73C6RT-rv: 5′-TCAATTATTGGACTGTGCT-3′; UBQ5-U: 5′-GTCCTTCTTTCTGGTAAACGT-3′, UBQ5-D: 5′-AACC CTTGAGGTTGAATCATC-3′). To compare relative amounts of transcripts in the samples, DNA fragments of the UBQ5 were first amplified and normalized sample volumes based on the amount of products corresponding to the UBQ5 transcripts were used for PCR.

Cloning of plant overexpression constructs and GUS-fusion constructs. For constitutive overexpression of c-Myc-tagged DOGT1 protein in Arabidopsis, the vector pBP1319 was constructed. It is derived from a modified version of the plant expression vector pPZP221 (25). The promoter cassette consisting of two copies of the 35S-promoter and the polyadenylation signal of CaMV strain Cabb B-D was used from the vector p2RT, a modified version of pRT100 (26).

The c-Myc-tagged DOGT1-fragment was released by SmaI+NotI digestion (Klenow filled) from the yeast expression vector and cloned into pBluescript SKII+, which was cut with ClaI+SmaI and treated with Klenow enzyme. In the next step, the gene was excised from the resulting plasmid as SalI+BamHI fragment and inserted in the XhoI+BamHI sites of p2RT. The obtained 2×35S c-Myc-DOGT1 cassette was isolated by PstI digestion and cloned into the unique PstI site of pPZP221 after the multiple cloning site in that vector had been destroyed by digesting the plasmid with EcoRI+SalI, filling the sites with Klenow and religating it. In the resulting vector pBP1319, the 2×35S c-Myc-DOGT1 cassette is orientated in the opposite direction than the 2×35S gentamycin resistance marker.

For construction of a transcriptional DOGT1-GUS fusion, the GUS vector pPZP-GUS.1 which originates from pPZP200 and contains the GUS gene from pBI101.1 (inserted HindIII+EcoRI into the MCS), was used (27). The DOGT1 promoter region was PCR amplified from genomic DNA using DNA polymerase with proof reading activity (Pfu Polymerase, MBI) and specific primers (DOGP-GUS-fw: 5′-GTTAAAAGCTTACATGTGCATTACGGTCTGTGTGAATA, DOGP-GUS-rv: 5′-TTTCGGATCCCATG ATTCAACCTTAGTAAGAAACTCTC). The resulting product was cloned in frame with the GUS gene HindIII+BamHI into the pPZP-GUS.1 vector. Constructs were confirmed by DNA sequencing.

Generation and analysis of transgenic A. thaliana. For all plant transformations, the recA deficient Agrobacterium tumefaciens strain UIA143 (28) which harbors the helper plasmid pMP90 (29) was used. A. thaliana was transformed applying the floral dip technique (30). The progeny of 15 independent transformants was selected through three generations to obtain homozygous lines.

For immunodetection of c-Myc-tagged DOGT1, about 200 mg-500 mg of plant material were homogenized in liquid nitrogen. 300 μl of extraction buffer (200 MM Tris-HCl pH 8.9; 200 mM KCl; 35 mM MgCl₂; 12.5 mM EGTA; 15 mM DTT; 0.6 mM sorbitol) and 15 μl of proteases inhibitor cocktail (Sigma, #9599) were added to the still frozen samples and the mixture was incubated under vigorous shaking for 15 minutes at 4° C. After centrifugation (14.000 rpm for 15 minutes at 4° C.), 200 μl of the supernatants were transferred into fresh tubes and stored at −20° C. Equivalent amounts of protein (50 μg) were used for Western blot analysis which was carried out using primary anti c-Myc antibody purified from hybridoma supernatant (clone 9E10).

For analysis of DON resistance, seeds of homozygous lines exhibiting high DOGT1-expression and Col-0 as control were germinated on MS media containing different concentrations of DON (5-30 ppm). Seedlings were grown for 5 weeks before the phenotype was documented. GUS activity was analyzed by staining seedlings or organs of adult plants in X-Gluc solution for 2 to 4 h at 37° C. (31).

### RESULTS

Isolation of the DON-glucosyltransferase (DOGT1) by heterologous expression in yeast. An unbiased functional screen based on heterologous expression of cDNAs in yeast was set up with the goal to identify plant genes that contribute to resistance against mycotoxins. Wild-type Saccharomyces cerevisiae is highly resistant to deoxynivalenol (DON). In order to reduce the amount of toxin necessary for the screen, a strain deficient in four ABC transporters was generated, which are to a large extent responsible for pleiotropic drug resistance in yeast (32). Strain YZGA452 (snq2Δ::hisG pdr5Δ::TRP1 pdr10Δ::hisG yor1Δ::hisG) is hypersensitive to a wide range of different xenobiotic substances and natural products, including DON. YZGA452 was transformed with a cDNA expression library of A. thaliana (20), where cDNAs are constitutively expressed under the control of the yeast phosphoglucerate kinase promoter. Ten million transformants were generated and diluted pools of transformants were plated on DON containing medium. After selection of DON resistant yeast colonies and confirmation of plasmid dependency of the phenotype, the insert was subcloned and sequenced.

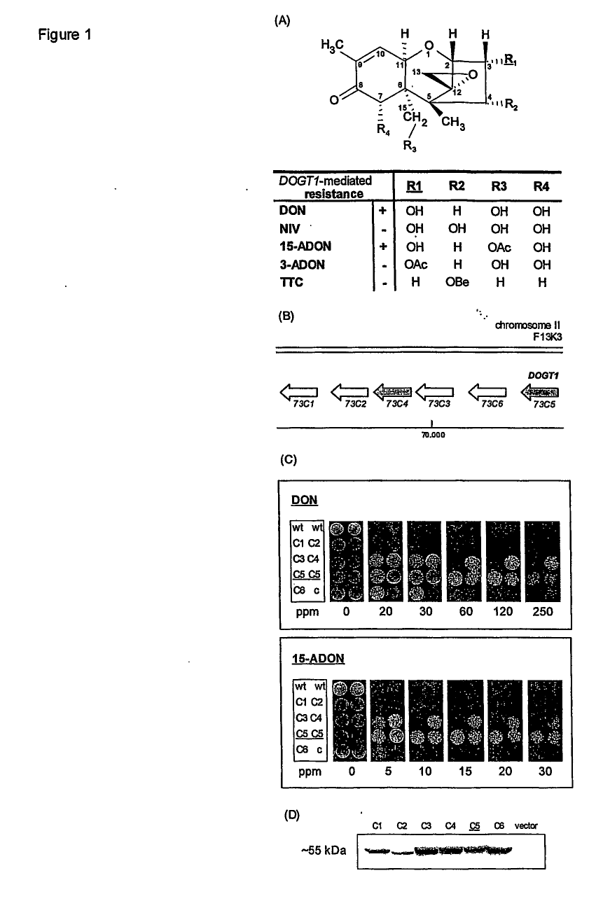

DOGT1 is a member of the UDP-glucosyltransferase family of A. thaliana and exhibits high similarity to salicylic acid and wound inducible genes of other species. The cDNA conferring resistance had a size of 1.75 kb and contained an open reading frame of 1488 bp length encoding a putative uridine diphosphate (UDP)-glucosyltransferase. The identified DON-glucosyltransferase (DOGT1) corresponds to gene UGT73C5 and belongs to subfamily 73C, part of group D of UDP-glucosyltransferases (UGTs) of A. thaliana (33). Arabidopsis UGTs constitute a very large gene family, that has been divided into 14 distinct groups, believed to have originated from common ancestors (17). DOGT1 is located in a cluster (FIG. 1B) together with five other members of subfamily 73C on chromosome II (BAC clone F13K3, At2g36800). All six tandemly repeated genes contain no introns, and are highly similar to each other (77-89% identity at the amino acid level). The similarity is also very high in the intergenic promoter regions.

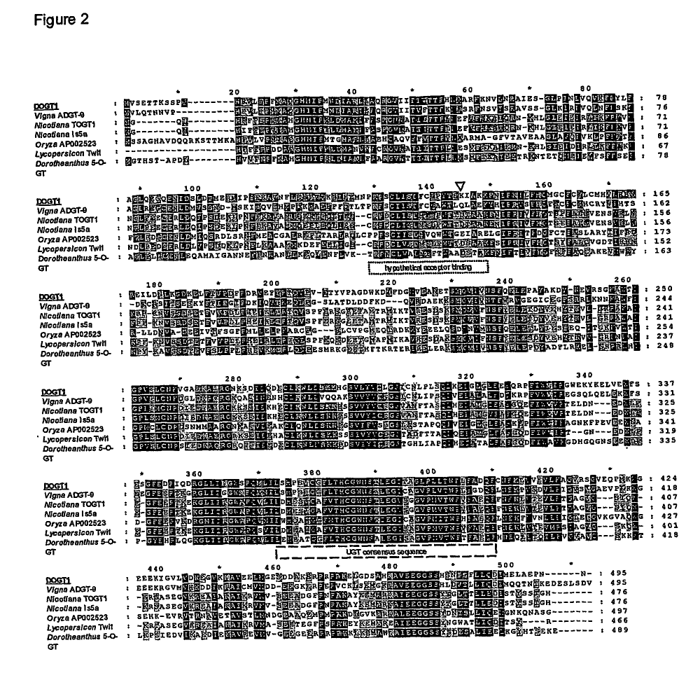

A database search with the DOGT1 amino acid sequence revealed high similarity to glucosyltransferases from tobacco (TOGT1, ref. 34; Is5a and Is10a, ref. 35) and tomato (Twi-1, ref. 36), the expression of which has been shown to be elevated following treatment with salicylic acid (SA), fungal elicitors or wounding (34, 36, 37), and to the betanidin 5-O-glucosyltransferase of Dorotheanthus bellidiformis (38). Two putative, uncharacterized glucosyltransferases from Vigna angularis (ADGT-9) and Oryza sativa with homology to DOGT1 were also included in the amino acid alignment shown in FIG. 2. Regions of high similarity were observed in both amino- and carboxy-terminal domains of the deduced amino acid sequences. Indicated in FIG. 2 are the hypothetical acceptor substrate binding region (38) and the UGT consensus sequence (33).

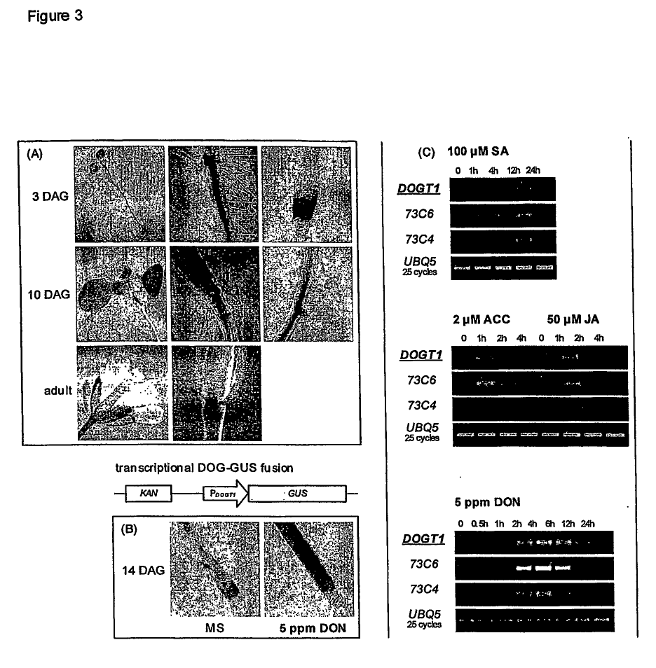

The expression of DOGT1 is developmentally regulated and induced by DON and other stress response related compounds. To investigate whether the expression of DOGT1 is regulated in a similar fashion as described for the related genes of other plant species, the ORF of the β-glucuronidase reporter gene was placed behind the DOGT1 promoter (P_{DOGT1}-GUS). The tissue specific expression of the transcriptional GUS fusion was examined histochemically in transgenic Arabidopsis homozygous for the fusion gene. The results shown in FIG. 3 demonstrate that DOGT1 expression is regulated developmentally and is overall rather low. In seedlings, GUS activity was observed to be root and hypocotyl specific, with the strongest expression in the vascular system, in meristematic tissue of the root tips (in the primary root as well as in lateral roots) and in the vasculature of the hypocotyl right after germination. Staining in the vasculature decreased significantly later in development and a patchy staining pattern appeared in epidermal root cells. In adult plants GUS activity was detected in late stages of flower development in petals and in abscission zones (FIG. 3A).

Exposure of seedlings to either DON (5 ppm for 4 h) or the ethylene precursor aminocyclopropylcarbonic acid (ACC, 2 μM for 1 h) was found to induce P_{DGT1}-GUS expression (FIG. 3). No induction of expression of the reporter was detected upon SA-treatment (200 μM for 12 h) or treatment with jasmonic acid (JA, 50 μM for 1 h). Semiquantitative reverse transcriptase PCR was applied to validate the results obtained from GUS reporter analyses by detecting changes in mRNA levels of DOGT1, 73C4 and 73C6 following treatment with the same concentrations of DON, SA, ACC and JA as used before.

As shown in FIG. 3C the results of the reverse transcriptase PCR confirm the DON inducible expression of DOGT1 previously observed with the reporter construct, and furthermore show that also the other two tested UGTs are DON inducible. An increase in the amounts of transcripts was observed already after 1 h of incubation with the toxin, reaching a peak after 4 h and declining again between 6 and 12 h. Interestinglu, 73C6 showed a stronger induction of expression by DON than DOGT1 or 73C4 although the data shown below indicate that it does not accept the toxin as a substrate. After SA treatment, DOGT1, 73C4 and 73C6 expression was evident at low levels at 4 h and slightly stronger at 12 h. It has to be noted that the applied concentration of 200 μM SA induced the expression of the 3 genes rather weakly. Jasmonic acid as well as treatment with ACC also lead to weak induction of expression of DOGT1 and UGT73CG apparent already after 1 h of treatment, but rapidly declining with no transcript accumulation detectable anymore after 2 h of exposure to the compounds (FIG. 3C).

Phenotypic determination of the spectrum of trichothecene resistance in yeast. Trichothecene toxicity in animals depends on the hydroxylation pattern, as well as on the position, number and complexity of esterifications (5). The basic trichothecene structure and numbering system of carbon atoms is shown in FIG. 1A. Members of the subclass B (e.g. DON and NIV), contain a keto group at carbon C-8, while type A trichothecenes, (e.g. the highly toxic T-2 toxin produced by F. sporotrichoides) do not. Extremely toxic are also macrocyclic trichothecenes, like veruccarin A, which contain a macrocyclic ring with ester bonds bridging carbon 4 and carbon 15. Yeast pdr5 mutants are hypersensitive to all the trichothecenes tested so far, allowing us to investigate the ability of DOGT1 and other genes in the cluster to confer resistance to various members of the trichothecenes.

The high similarity of UGT73C1, C2, C3, C4, C5 (DOGT1) and C6 and their clustered appearance on the chromosome suggests that they have evolved via gene duplication from one ancestral gene and might therefore have related enzymatic properties. To analyze possible similarities in their function, the six ORFs were amplified with specific primers and expressed in yeast as fusion proteins with an N-terminal c-Myc epitope tag. Comparison of transformants expressing tagged or untagged DOGT1 showed that the epitope did not interfere with the DON-protective activity. The yeast transformants representing the full gene set of the cluster were spotted on media containing increasing concentrations of various trichothecenes. Transformants containing the empty expression vector were used as controls.

The results shown in FIG. 1 revealed that UGT73C4, like DOGT1, confers DON resistance. Although C4 has only the second highest similarity (79% identity to DOGT1), it had the ability to detoxify DON and 15-ADON (FIG. 1C), but failed to confer resistance to 3-ADON, NIV, TTC, HT-2-toxin, T2-toxin, DAS and VA. In contrast, UGT73C6 which exhibits with 89% identity on amino acid level the highest similarity did not protect against any of the tested trichothecenes. Likewise, 73C3 was not able to confer resistance to the tested mycotoxins although all of the four proteins were expressed to a similar extent in yeast. The 73C1 and 73C2 constructs did also not enhance toxin resistance, however this could also be due to lower recombinant protein levels present in the yeast transformants (FIG. 1D).

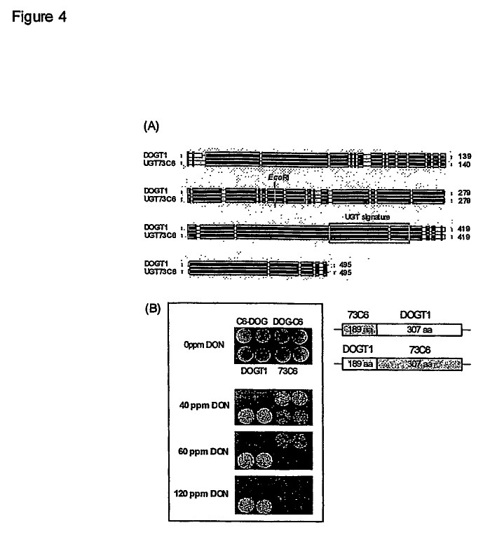

DON detoxification specificity is located in the N-terminal part of DOGT1. The different characteristics of the closely related enzymes despite their high sequence similarity opened the possibility to elucidate features of the proteins which are crucial for DON recognition. An EcoRI site present in both DOGT1 and 73C6 (see FIG. 4A) was used to construct hybrids containing the N-terminal part of one and the C-terminal part of the respective other gene. Expression in yeast and exposure to DON containing media revealed that the N-terminus of DOGT1 (consisting of the first 189 aa) is essential for the ability to detoxify DON (FIG. 4B).

A lysine residue at position 136 in DOGT1 is conserved in the detoxifier 73C4 but replaced by other amino acids in the two nondetoxifying homologues C3 and C6. Site directed mutagenesis was used to introduce mutations into DOGT1 and the UGT73C6 leading to a change of the lysine residue 136 in DOGT1 to glutamic acid, which is present at the corresponding position in C6 (amino acid 137). The mutant DOGT^{K136E} protein protected with the same efficiency as DOGT1, demonstrating that K136 is not essential for the DON detoxification ability. Also the reverse construct 73C6^{E137K} remained inactive.

In vivo and in vitro analysis proof that DOGT1 catalyses the transfer of glucose from UDP-glucose specifically to the 3-OH position of DON. The protection against 15-ADON and the inability to confer resistance against 3-ADON suggested that DOGT1 may catalyse the formation of DON-3-O-glucoside. To test this hypothesis, first chemically synthesized DON derivatives with the glucose moiety was attached either to the C3- or C15-hydroxyl group of DON. The two products were characterized with LC-MS/MS.

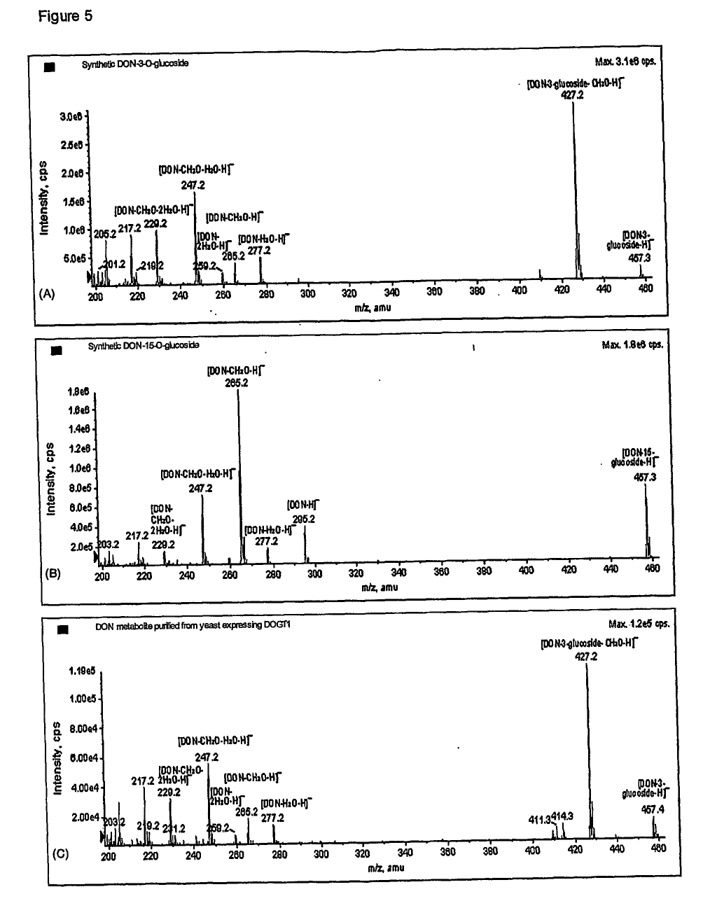

The DON-3-O-glucoside and DON-15-O-glucoside eluted at 12.43 min and 12.68 min, respectively. The mass spectra of the glucosides showed characteristic differences in their fragmentation pattern (FIG. 5). While the DON-3-O-glucoside fragmented to an ion of 427.2 m/z under the given conditions, the same ion was not detected with DON-15-O-glucoside. The loss of 30 atomic mass units can be explained by the cleavage of the —CH₂OH group at C₆, which is prevented when the hydroxyl group is conjugated with glucose as in DON-15-O-glucoside. Further breakdown (MS) of the DON-glucosides in the linear ion trap showed an almost identical fragmentation pattern as that of the [DON-H]-ion (295.3 m/z, not fragmented in Q2), confirming the presence of DON entities in the reaction products.

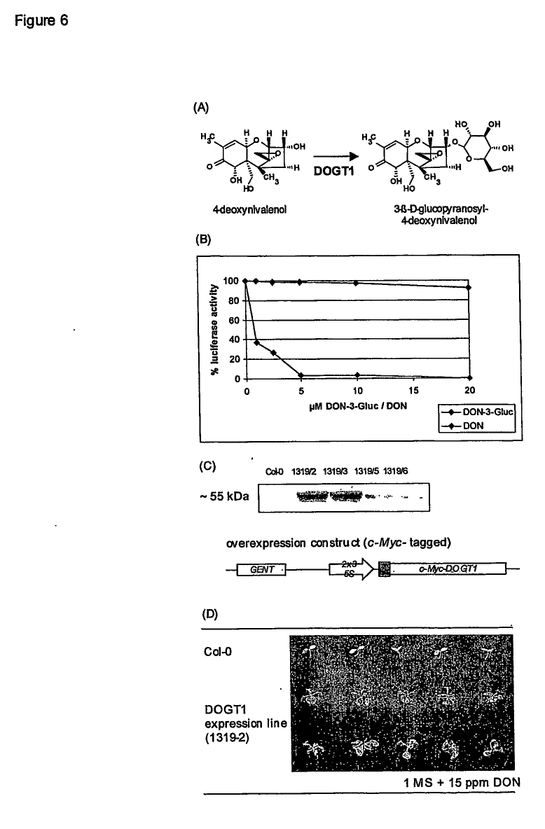

With these tools at hand it was possible to directly determine which glucoside was formed in yeast cells. Yeast strain YZGA515, incapable of converting DON into 3-ADON due to a deletion of the yeast acetlytransferase gene AYT1, was transformed with both the DOGT1 expression vector and a plasmid containing a gene encoding a trichothecene insensitive mutant ribosomal protein L3, the target of trichothecenes, to increase DON tolerance of yeast cells. After the resulting strain had been incubated with high amounts of DON, reaching a final concentration of 1000 ppm in the medium, the DON-metabolite was extracted from the cells and identified as the expected 3-O-glucopyranosyl-4-deoxynivalenol (FIG. 6A) by HPLC and mass spectroscopy. FIG. 5 shows the fragmentation pattern of the synthesized reference substances and the product peak from yeast expressing DOGT1. As expected, the metabolite was not present in the control strain, lacking DOGT1 activity.

In order to further verify substrate specificity, a GST-DOGT1 fusion was constructed. The GST-DOGT1 fusion gene was also expressed in yeast, where it conferred DON resistance like wild-type DOGT1. The gene product was expressed in E. coli and affinity purified. The reaction products generated in vitro during incubation of either the DOGT1 fusion-protein or GST with UDP-[¹⁴C] glucose and DON were analyzed using TLC. A spot with the same Rf value as the synthesized DON-glucoside was observed in the reaction containing the GST-DOGT1 fusion protein but not in the control.

Glucosylation of 4-deoxynivalenol severely reduces its toxicity. To analyze whether the 3-β-D-Glucopyranosyl-4-deoxynivalenol is less phytotoxic than DON, a wheat germ extract coupled in vitro transcription/translation system was used. As shown in FIG. 6B the presence of 1 μM DON in the reaction inhibited protein translation significantly, leading to 36.8% reporter enzyme activity compared to the control (100%). 5 μM of the toxin resulted in only 3.1% luminescence remaining whereas 20 μM of the synthesized DON-3-glucoside only inhibited luciferase activity by 8%. These results proof that glucosylation of DON is a detoxification process.

Overexpression of DOGT1 in Arabidopsis thaliana increases DON resistance. Transgenic A. thaliana constitutively expressing DOGT1 under the control of a tandem 35S promoter were generated and the amount of recombinant protein in transformants determined by Western blotting utilizing the N-terminal c-Myc epitopetag. Seeds of the homozygous line 1319/2, which was found to have a high DOGT1 expression level (FIG. 6C), and wild-type Col-0 as control were germinated on MS media containing 5-30 ppm DON and grown for 5 weeks before the phenotype was analyzed.

The main phytotoxic effect observed was slow germination of wild-type plants. Root formation did not occur at all, cotyledons developed but started to bleach before true leaves could be formed. After 3 weeks of exposure to DON most of the wild-type seedlings had lost all green color and ceased growth. DOGT1-overexpression lines showed also a clear delay in development. Compared to wild-type, germination occurred earlier, roots were formed, cotyledons did not bleach and true leaves appeared. The differences in DON sensitivity observed were most apparent on media containing 15 ppm of toxin (FIG. 6D). Control transformants with empty vector (containing solely the gentamycin resistance gene), and lines expressing low amounts of DOGT1 were as sensitive as wild-type Col-0.

### DISCUSSION

Biotechnological relevance. Fusarium diseases of wheat and barley are of high economic significance for countries around the world. The United States for instance have been severely hit by Fusarium epidemics in the last decade. Direct losses due to FHB for the US wheat producers have been estimated to average about $260 million annually and over the period 1998-2000 the combined economic losses for small grain cereals were estimated at $2.7 billion (39). Deoxynivalenol contamination of large portions of the harvest can lead to high intake of the mycotoxin. Children are the population group most at risk to exceed the tolerable daily intake level (TDI) for DON. In the problematic year 1998 80% of 1 year old children in the Netherlands exceeded the TDI (7).

The high importance of Fusarium diseases justifies research on mycotoxin inactivation, although resistance against the pathogen is most likely a polygenic trait in crop plants and toxin resistance just one of its components. The production of trichothecenes is the only virulence factor of the fungal pathogen which is experimentally confirmed so far, with the exception of pleiotropic mutations in MAP kinases affecting not exclusively virulence but also conidiation, perithecia formation, vegetative growth and mycotoxin production (40, 41). The present finding that overexpression of DOGT1 leads to increased deoxynivalenol resistance in transgenic Arabidopsis opens new possibilities for biotechnological approaches aiming to antagonize the fungal virulence factor. The only result published so far relies on a Fusarium acetyltransferase (42) converting DON into 3-ADON, which is about 2-fold less toxic to laboratory animals and therefore no detoxification within the meaning of the present invention. The phenotype of transformed yeast and Arabidopsis, as well as data obtained using in vitro translation assays, demonstrate that the DON-glucoside exhibits reduced toxicity. In general, glucosylation converts reactive and toxic aglucones into stable and non-reactive storage forms, thereby limiting their interaction with other cellular components. The addition of a sugar blocks the reactive site of the substance and consequently reduces toxicity for the plant. Such modifications are furthermore considered to provide access to membrane-bound transporters opening exit pathways from the cytosol to for example the cell wall or the vacuole (43).

Glucosylation of trichothecenes represents a detoxification process for plants. Yet, in the digestive tract of humans and animals the mycotoxin-glucoconjugates could be easily hydrolyzed, regenerating the toxin. The extent to which the DON-glucoside is transported to the vacuole or to the apoplast is currently unknown. Before one attempts to use transgenic plants overexpressing a DON-glucosyltransferase, it should be investigated whether only the vacuolar glucoside or also toxin bound to cell wall material as “insoluble residue” is a source of “masked mycotoxin” (44), which escapes traditional analytical techniques and may be of much higher toxicological relevance as currently assumed. The analytical tools and reference materials developed during this work is suitable to address these questions.

Inactivation of toxins by glucosylation seems to be a prominent natural mechanism for resistance, enabling plants to cope with the enormous diversity of toxic microbial metabolites they may encounter in nature. DOGT1 is one of 118 UGT genes in A. thaliana which are predicted to conjugate small molecules.

The expression of UGT genes in yeast is a valuable complementary approach to the widely used expression in E. coli, especially when the respective chemicals have targets in eukaryotes only. One problem is that wild-type yeast cells are frequently “impermeable” to the substances of interest. Inactivating several ABC-transporters in the present host strain was a prerequisite for the approach to select resistance conferring cDNAs. In the case of trichothecenes this allowed the phenotypic detection of the activity of the expressed genes in a simple plate assay. In principle, this approach could be adopted for many other substances.

Gene evolution and substrate specificity. DOGT1 is located in a cluster (FIG. 1B) with 5 other members of family 73C on chromosome II (At2g36800) indicative for gene duplication by unequal recombination events (32). One could speculate that such a gene amplification event provides a selective advantage under toxin stress. DOGT1 and other members in the cluster have very similar protein sequences suggesting redundancy in enzymatic function. However, when testing the homologues for detoxification of trichothecenes, it was found that yeast expressing the gene with the highest sequence similarity UGT73C6 was as sensitive as wild-type to all toxins tested (as well as UGT73C3), while the second closest homologue UGT73C4 exhibited the same properties as DOGT1. Enzymatic activity towards different hydroxycoumarins has been shown for all 73C cluster members (45), indicating that the UGTs lacking DON-protective activity are not simply loss of function alleles. The cluster is therefore an example that supports the hypothesis that duplicated UGT genes can acquire new substrate specificities and functions during evolution.

Plant UDP-glucosyltransferases are structurally very similar in their carboxy-terminal signature motif to mammalian UGTs which use UDP-glucuronic acid instead of UDP-glucose as donor substrate. The mammalian enzymes play a central role in metabolism and detoxification of chemicals like carcinogens or hydrophobic drugs. Higher plant UGTs have been found to be involved in a parallel range of activities also modifying xenobiotic substances such as herbicides and other pesticides (46, 47). [One open question is whether these detoxification reactions are side-activities of UGTs conjugating endogenous plant compounds. Although recent publications are in favor of the hypothesis that UGTs responsible for the conversion of endogenous substrates may also account for the capacity of plants to detoxify xenobiotic substances (48) this question will need further work to be solved.]

The finding that DOGT1 protects against DON but not NIV, which possesses one additional hydroxyl group at residue 4 (FIG. 1A), indicates that the variation of the hydroxylation pattern of the trichothecene backbone might be an important mechanism of the fungus to escape such detoxification reactions. It is unknown whether plants can also conjugate NIV, which has higher toxicity than DON in animal systems (5). The lack of activity against NIV stringlu argues against use of a single DOGT1-like gene in transgenic plants, as NIV producing chemotypes of F. graminearum could get a selective advantage.

The construction of hybrid genes by shuffling the N- and C-terminal part of DOGT1 and UGT73C6 and their expression in yeast proved that the N-terminal 189 amino acids of DOGT1 are essential for its ability to detoxify DON, in agreement with the hypothesis that substrate binding may occur in a conserved hydrophobic area between amino acid 130 and 150 (38). Comparison of two genes with protective effect and two genes without activity, but similar expression level, suggested that a lysine, which is present at amino acid position 136 in DOGT1 (and at the corresponding position in UGT73C4) but replaced in the two UGT's unable to detoxify trichothecenes, could be important for DON detoxification. This hypothesis was falsified using site directed mutagenesis and expression of the engineered proteins in yeast.

Information on specific domains or amino acids involved in acceptor binding of DOGT1 may be potentially useful to search for glucosyltransferases capable to detoxify DON in crop plants like wheat or maize. It should be considered though that attempts to assign functions to UGTs based exclusively on amino acid similarity are highly error prone, as shown in the case of the 73C cluster. Nevertheless, the functionality of candidate genes can be easily tested by expression in yeast. One possibility which can not be excluded is that glucosides of trichothecenes are formed in crop plants by structurally fairly dissimilar UGTs. For instance, UGT84B1 was shown to exhibit the highest in vitro activity against the auxin indole-3-acetic acid (IAA) of all A. thaliana UGTs tested, yet this enzyme does not display the highest sequence similarity to the functional homologue in maize, the iaglu gene (49).

Regulation of gene expression. Genes with high sequence similarities to DOGT1 from tobacco and tomato have been shown to be induced by salicylic acid or wounding (34, 36, 37). The analysis of expression of DOGT1 and 73C6 following SA and JA treatment showed that they responded weekly with elevated mRNA levels to SA, JA and the ethylene precursor ACC. Inducibility of gene expression by SA, JA or ethylene is considered to be indicative for a possible role of the upregulated gene product in plant stress or defense responses (50).

Using analysis of mRNA levels and a GUS-reporter construct it was possble to show that DOGT1 transcription in wild-type A. thaliana is developmentally regulated and is rapidly and significantly induced in response to DON exposure. Induction of expression by the mycotoxin has also been observed for other 73C cluster members, regardless of their DON-protective activity. It would be interesting to clarify whether the inducibility is compound specific or a general phenomenon for protein biosynthesis inhibitors. The “ribotoxic stress response” is currently actively investigated by researchers in human cells (51), where the mycotoxin induces the expression of cyclooxigenase-2, a key enzyme in the synthesis of mediators of the inflammatory response, via MAP kinase mediated signaling. The DON inducible expression of DOGT1 is an attractive starting point for investigating whether similar mechanisms exist in plants.

With the present invention it was shown that the huge gene family of glucosyltransferases, especially—as shown in the examples—UGTs plays an important role in plant pathogen interaction by participating in detoxification of metabolites produced by microbes to increase their virulence on hosts. Similar to the large family of leucine-rich-repeat type resistance genes (52) involved in pathogen recognition, gene amplification and diversifying selection of UGTs could lead to a broad spectrum protection against fungal toxins. The selective pressure to escape such glucosylation reactions may be a driving force in evolution of microbial biosynthetic reactions leading to a wide spectrum of toxin structures, as observed for trichothecenes.

### REFERENCES

1. McMullen, M. P., Jones, R. and Gallenberg, D. (1997) Plant Dis. 81, 1340-1348

2. Grove, J. F. (1996) Progress in the Chemistry of Organic Natural Products, pp. 1-70, Springer, Wien

3. Cundliffe, E. and Davies, J. E. (1977) Agents Chemother. 11, 491-499

4. O'Donnel, K., Kistler, H. C., Tacke, B. K. and Casper, H. H. (2000) PNAS 97, 7905-7910

5. Betina, V. (1989) Mycotoxins—chemical, biological and environmental aspects. pp. 192-241, Elsevier, Amsterdam

6. Canady, R. A., Coker, R. D., Egan, K., Krska, R., Kuiper-Goodman, T., Olsen M., Pestka, J. , Resnik, S., & Schlatter, J. (2001) WHO Food Additives Series 47, 419-528

7. Codex Committee on Food Additives and Contaminants (2002) Joint FAO/WHO Food Standards Programme, CX/FAC 03/35, www.codexalimentarius.net

8. McCormick, S. P. (2003) Fusarium head blight of wheat, pp. 165-183, American Phytopathological Society, St. Paul, Minn., USA

9. Desjardins, A. E., Procter, R. H., Bai, G., McCormick, S. P., Shaner, G., Buechley, G. and Hohn, T. (1996) Mol. Plant-Microbe Interact. 9, 775-781

10. Bai, G. H, Desjardins, A. E. and Plattner R. D. (2002) Mycopathologia 153, 91-98

11. Kang, Z. and Buchenauer, H. (1999) Physiol. Molec. Plant Pathol. 55, 275-288

12. Mesterhazy, A. (2003) Fusarium head blight of wheat, pp. 363-380, American Phytopathological Society, St. Paul, Minn., USA

13. Coleman, J. O. D., Blake-Kalff, M. M. A. and Davies, T. G. E. (1997) Trends Plant Sci. 2, 144-151

14. Scott, P. M., Nelson, K., Kanhere, S. R., Karpinski, K. F., Hayward, S., Neish, G. A. and Teich, A. (1984) Appl. Environ. Microbiol. 48, 884-886

15. Miller, J. D. and Arnison, P. G. (1986) Can J Plant Path 8, 147-150

16. Sewald, N., von Gleissenthall, J. L., Schuster, M., Muller, G. and Aplin, R. T. (1992) Tetrahedron Asymmetry 3, 953-960

17. Ross, J., Yi, L., Lim, E.-K. and Bowles, D. (2001) Genome Biol. 2, 3004.1-3004.6.

18. Sikorski, R. S. and Hieter, P. (1989) Genetics 122, 19-27

19. Murashige, T. and Skoog, F. (1962) Plant Physiol. 15, 473-497

20. Minet, M., Dufour, M.-E. and Lacroute, F. (1992) Plant J. 2, 417-422

21. Ballester, R., Michaeli, T., Ferguson, K., Xu, H.-P., McCormick F, and Wigler, M. (1989), Cell 59, 681-686

22. Egner, R., Mahe, Y., Pandjaitan, R. and Kuchler, K. (1995) Mol Cell Biol 15, 5879-5887

23. Pogulis, R. J., Vallejo, A. N. and Pease, L. R. (1996) Methods Mol Biol 57, 167-176

24. Savard, M. (1991) J. Agr. Food Chem., 39, 570-574

25. Hajdukiewicz, P. Svab, Z. and Maliga, P. (1994) Plant Mol Biol 25, 989-994

26. Topfer, R., Matzeit, V., Groneborn, B., Schell, J. and Steinbiss H.-H. (1987) Nucleic Acid Research 14, 5890

27. Diener, A. C., Li, H., Zhou, W., Whoriskey, W. J., Nes, W. D. and Fink, G. R. (2000) Plant Cell 12, 853-870

28. Farrand, S. K., O'Morchoe, S. P. and McCutchan, J. (1989) J Bacteriol 171, 5314-5321

29. Koncz, C. and Schell, J. (1986) Mol Gen Genet 204, 383-396

30. Clough, S. J. and Bent, A. F. (1998) Plant J. 16, 735-743

31. Jefferson, R. A. (1987) Plant Mol. Biol. Rep. 5, 387-405

32. Wolfger, H., Mamnun, Y. M. and Kuchler, K. (2001) Res Microbiol. 152, 375-89

33. Li, Y., Baldauf, S., Lim, E.-K., and Bowles, D. J. (2001) J. Biol Chem 276, 4338-4343

34. Fraissinet-Tachet, L., Baltz, R., Chong, J., Kauffmann, S., Fritig, B. and Saindrenan, P. (1998) FEBS 437, 319-323

35. Horvath, D. M. and Chua, N.-H. (1996) Plant Mol Biol 31, 1061-1072

36. Truesdale, M. R., Doherty, H. M., Loake, G. J., McPherson, M. J., Roberts, M. R. and Bowles, D. J. (1996) Plant Physiol 112, 446

37. Horvath, D., Huang, D. J. and Chua, N.-H. (1998) Mol. Plant-Microbe Interact. 11, 895-905

38. Vogt, T., Grimm, R. and Strack, D. (1999) Plant J 19, 509-519

39. Nganje, W. E., Johnson, D. D., Wilson, W. W., Leistritz, F. L., Bangsund, D. A. and Tiapo, N. M. (2001) Agribusiness and Applied Economics Report 464

40. Jenczmionka, N. J., Maier, F. J., Losch, A. P. and Schafer, W. (2003) Curr. Genet. 43, 87-95

41. Hou, Z., Xue, C., Peng, Y., Katan, T., Kistler, H. C. and Xu, J. R. (2002) Mol Plant Microbe Interact. 15, 1119-1127

42. Okubara, P. A., Blechl, A. E., McCormick, S. P., Alexander, N. J., Dill-Macky, R. and Hohn, T. M. (2002) Theor Appl Genet. 106, 74-83

43. Jones, P. and Vogt, T. (2001) Planta 213, 164-174

44. Engelhardt, G., Ruhland, M. and Wallnöfer, P. R. (1999) Adv. Food Sci. 21, 71-78

45. Lim, E.-K., Baldauf, S., Li, Y., Elias, L., Worrall, D., Spencer, S. P., Jackson, R. G., Taguchi, G., Ross, J. and Bowles, D. (2003) glucobiol. 13, 139-145

46. Wetzel, A. and Sandermann, H. (1994) Arch. Biochem. Biophys. 314, 323-328

47. Leah, J. M., Worral, T. L. and Cobb, A. H. (1992) Pest. Science 34, 81-87

48. Meβner, B., Thulke, O. and Schäffner, A. R. (2003) Planta 217, 138-146

49. Jackson, R. G., Lim, E.-K., Li, Y., Kowalczyk, M., Sandberg, G., Hoggett, J., Ashford, D. A. and Bowles, D. (2001) J. Biol. Chem. 276, 4350-4356

50. Reymond, P. and Farmer, E. E. (1998) Curr Opin Plant Biol. 1, 404-411

51. Laskin, J. D., Heck, D. E. and Laskin, D. L. (2002) Toxicol Sci. 69, 289-291

52. Meyers, B. C., Kozik, A., Griego, A., Kuang, H. and Michelmore, R. W. (2003) Plant Cell 15, 809-834

53. Eudes F., Comeau, A., Rioux S. and Collin J. (2000) Can. J. Plant Path. 22, 286-292

54. Potrykus, I. and Spangenberg, G. (Eds.): Gene Transfer to Plants. Springer Verlag Berlin (1995), ISBN 3-54058406-4

Abbreviations:

FHB, Fusarium head blight; DON, deoxynivalenol; 15-ADON, 15-acetyl-deoxynivalenol; NIV, nivalenol; UGT, UDP-glucosyltransferase; ABC, ATP-binding cassette; DOGT1, DON-glucosyltransferase; ORF, open reading frame; GUS, β-glucuronidase; SA, salicylic acid; JA, jasmonic acid; ACC, 1-aminocyclopropylcarbonic acid; TDI, tolerable daily intake; IAA, indole-3-acetic acid; DAG, days after germination.

1.-19. (canceled)

20. A method comprising contacting a mycotoxin with a glucosyltransferase in the presence of an activated glucose.

21. The method of claim 20, wherein the mycotoxin is glucosylated.

22. The method of claim 20, wherein the mycotoxin is detoxified.

23. The method of claim 20, wherein the glucosyltransferase is UDP-glucosyltransferase and the activated glucose is UDP-glucose.

24. The method of claim 20, wherein the mycotoxin is a trichothecene.

25. The method of claim 24, wherein the trichothecene has a free hydroxy-group at position C₃ and two hydrogen groups at C₄.

26. The method of claim 20, wherein the mycotoxin is deoxynivalenol, 15-acetyldeoxynivalenol, and/or 15-Acetoxyscirpendiol.

27. The method of claim 20, wherein the method is performed in vivo using recombinant DNA technology and/or glucosyltransferase expression enhancing compounds.

28. The method of claim 27, wherein the glucosyltransferase is expressed in a transgenic plant cell containing a recombinant glucosyltransferase and/or a recombinant regulating region for a glucosyltransferase.

29. The method of claim 27, wherein a mycotoxin-inducible promoter of a glucosyltransferase is used.

30. The method of claim 20, wherein the glucosyltransferase is immobilised to a solid surface and a mycotoxin containing solution or suspension is contacted with said immobilised glucosyltransferase.

31. The method of claim 20, wherein the glucosyltransferase is a UDP-glucosyltransferase corresponding to subfamily 73C of Arabidopsis thaliana.

32. The method of claim 31, wherein the glucosyltransferase is UDP-glucosyltransferase 73C5 and 73C4.

33. The method of claim 20, further defined as a method for detoxification of trichothecenes in agriculture or beer production.

34. A recombinant cell being resistant to mycotoxins comprising a heterologous glucosyltransferase or an enhanced expression activity of an endogeneous glucosyltransferase due to transgenic expression regulating elements.

35. The cell of claim 34, further defined as being resistant to trichothecenes.

36. The cell of claim 34, further defined as a plant cell or a yeast cell.

37. The cell of claim 34, further comprising at least one ABC transporter which has reduced activity or is inactive.

38. The cell of claim 37, wherein the at least one ABC transporter has at least one pdr5 deletion.

39. The cell of claim 34, further comprising three or more ABC transporters which have reduced activity or are inactive.

40. The cell of claim 39, wherein the three or more ABC transporters have at least one pdr5 deletion.

41. The cell of claim 37, further defined as comprising a deletion in pdr5, pdr10, snq2, yor1, pdr15, and/or ayt1.

42. The cell of claim 34, further defined as resistant to Fusarium.

43. A plant comprising the recombinant cell of claim 34.

44. The plant of claim 43, further defined as resistant to Fusarium.

45. A method for detoxification of mycotoxins in mycotoxin-ontaining solutions or suspensions comprising obtaining a cell of claim 34 and adding it to the solution.

46. The method of claim 45, further defined as a method for detoxification of trichothecenes in agriculture or beer production.
